(19) United States
(12) Patent Application Publication
(10) Pub. No.: US 2014/0343298 A1
(43) Pub. Date: Nov. 20, 2014

(54) METHOD FOR PRODUCING GLYCOLIDE

(71) Applicants: Haruyasu Yamaji, Tokyo (JP); Kazuyuki Yamane, Tokyo (JP); Shigeru Suzuki, Tokyo (JP)

(72) Inventors: Haruyasu Yamaji, Tokyo (JP); Kazuyuki Yamane, Tokyo (JP); Shigeru Suzuki, Tokyo (JP)

(73) Assignee: KUREHA CORPORATION, Tokyo (JP)

(21) Appl. No.: 14/344,199

(22) PCT Filed: Sep. 10, 2012
(86) PCT No.: PCT/JP2012/073087

§ 371 Date: Apr. 22, 2014

(30) Foreign Application Priority Data
Sep. 12, 2011 (JP) 2011-198627

(51) Int. Cl. C07D 319/12

(52) U.S. Cl. CPC C07D 319/12
USPC 549/274

(57) ABSTRACT

A method for producing glycolide comprises depolymerizing a glycolic acid oligomer in the presence of a phenol-based antioxidant.

### TECHNICAL FIELD

The present invention relates to a method for producing glycolide, and more specifically to a method for producing glycolide obtained by depolymerizing a glycolic acid oligomer.

### BACKGROUND ART

Polyglycolic acid is a resin material having excellent biodegradability, gas-barrier property, strength, and the like, and has been used, in a wide range of technical fields, as medical polymer materials such as surgical sutures and artificial skins; packaging materials such as bottles and films; and a resin material for various industrial products such as injection molded articles, fibers, vapor-deposited films, and fishing lines.

Polyglycolic acid can be obtained by dehydration polycondensation of glycolic acid. However, the polyglycolic acid obtained by this method has a low degree of polymerization and a weight average molecular weight of 20 thousand or less. Although this polyglycolic acid has an excellent biodegradability, characteristics such as gas-barrier property, strength, and durability of the polyglycolic acid are not sufficiently satisfactory for many fields.

For this reason, polyglycolic acid is generally produced by ring-opening polymerization of glycolide. This method makes it possible to easily control the degree of polymerization of polyglycolic acid, and to obtain polyglycolic acid having a high degree of polymerization and a weight average molecular weight exceeding 20 thousand. The glycolide used here is generally synthesized as follows. Specifically, glycolic acid is subjected to dehydration-polycondensation according to the following formula (I) to thereby synthesize a glycolic acid oligomer having a low degree of polymerization:

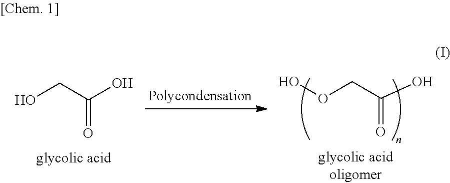

Next, the glycolic acid oligomer is depolymerized according to the following formula (II):

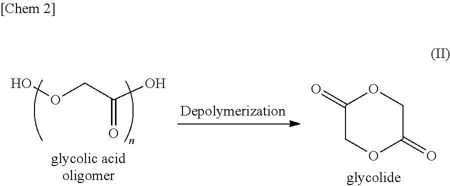

Here, in the above-described depolymerization reaction of a glycolic acid oligomer, the heavy-component formation from the oligomer due to the heating has been conventionally regarded as a problem, and various improvements have been made to the method for producing glycolide utilizing the depolymerization reaction. For example, Japanese Unexamined Patent Application Publication No. Hei 9-328481 (PTL 1) and International Publication No. WO02/014303 (PTL 2) disclose that the heavy-component formation from the glycolic acid oligomer can be suppressed by conducting the depolymerization reaction of the oligomer in a specific high-boiling point polar organic solvent. However, even in these methods, the heavy-component formation from the glycolic acid oligomer occurs when the depolymerization reaction is conducted repeatedly. Hence, a further improvement has been required.

### CITATION LIST

### Patent Literature

[PTL 1] Japanese Unexamined Patent Application Publication No. Hei 9-328481
[PTL 2] International Publication No. WO02/014303

### SUMMARY OF INVENTION

### Technical Problem

The present invention has been made in view of the above-described problem of the conventional technologies, and an object of the present invention is to provide a novel method for producing glycolide in which the heavy-component formation from the glycolic acid oligomer can be suppressed in a case where glycolide is produced for a long period by utilizing depolymerization reaction of the oligomer.

### Solution to Problem

The present inventors have conducted earnest study to achieve the above object. As a result, the present inventors have found that the heavy-component formation from the glycolic acid oligomer can be suppressed by conducting depolymerization of the oligomer in the presence of an antioxidant, so that glycolide can be produced for a long period. This finding has led to the completion of the present invention.

Specifically, a method for producing glycolide of the present invention comprises depolymerizing a glycolic acid oligomer in the presence of a phenol-based antioxidant. The phenol-based antioxidant is preferably a phenol-based antioxidant having a molecular weight of 300 or higher.

In the method for producing glycolide of the present invention, the depolymerization of the glycolic acid oligomer is preferably conducted in a solvent, and the solvent is preferably a high-boiling point polar organic solvent having a boiling point of 230 to 450° C. In addition, the glycolide obtained by the depolymerization in the solvent is preferably co-distilled off with the solvent. Moreover, in the method for producing glycolide of the present invention, it is also preferable to conduct the depolymerization of the glycolic acid oligomer in the presence of a tin compound.

### Advantageous Effects of Invention

According to the present invention, the heavy-component formation from a glycolic acid oligomer can be suppressed in a case where the glycolide is produced by utilizing depolymerization reaction of the oligomer, so that glycolide can be produced for a long period.

### DESCRIPTION OF EMBODIMENTS

Hereinafter, the present invention will be described in detail based on preferred embodiments thereof.

A method for producing glycolide of the present invention comprises depolymerizing a glycolic acid oligomer in the presence of a phenol-based antioxidant. In the present invention, the glycolic acid oligomer is preferably depolymerized in a solvent, in the presence of a tin compound, or in the presence of a solubilizing agent, or under a condition where two or more thereof are combined.

(1) Glycolic Acid Oligomer

The glycolic acid oligomer used in the present invention is a polyglycolic acid having a weight average molecular weight of 20 thousand or less. The glycolic acid oligomer can be synthesized by a polycondensation reaction of glycolic acid. Here the weight average molecular weight of the glycolic acid oligomer is a value measured by gel permeation chromatography (GPC) with hexafluoroisopropanol as an eluent and converted to that of standard polymethyl methacrylate.

An example of a method for synthesizing the glycolic acid oligomer is described below. However, the glycolic acid oligomer used in the present invention is not limited to glycolic acid oligomers synthesized by this method. For example, the glycolic acid oligomer is obtained as follows. Specifically, a polycondensation reaction or an ester-exchange reaction is conducted by heating at least one of glycolic acid, esters thereof (for example, lower alkyl esters) and salts thereof (for example, sodium salt), when necessary, in the presence of a polycondensation catalyst or an ester-exchange catalyst, at a temperature of usually 100 to 250° C., and preferably 140 to 230° C., until substantially no further low-molecular weight substances such as water and alcohol are distilled off. While the thus obtained glycolic acid oligomer may be used directly as a raw material in the production method of the present invention, the thus obtained glycolic acid oligomer is preferably used as the raw material after unreacted materials, components having low degrees of polymerization, the catalyst, and the like are removed by washing with a poor solvent such as benzene or toluene.

The degree of polymerization of the glycolic acid oligomer used in the present invention is not particularly limited. The glycolic acid oligomer preferably has such a degree of polymerization that the melting point (Tm) of the glycolic acid oligomer is 140° C. or higher (more preferably 160° C. or higher, and particularly preferably 180° C. or higher). If the melting point of the glycolic acid oligomer is lower than the lower limit, the yield of the glycolide obtained by the depolymerization reaction tends to be low. Here the melting point of the glycolic acid oligomer is a temperature detected as an endothermic peak temperature which is observed when a calorimetric analysis is conducted by using a differential scanning calorimeter (DSC) under an inert gas atmosphere and under a condition of a rate of temperature rise of 10° C./minute. An upper limit value of the melting point of the glycolic acid oligomer is approximately 220° C.

(2) Antioxidant

The antioxidant used in the present invention is a phenol-based antioxidant. By depolymerizing the glycolic acid oligomer in the presence of the phenol-based antioxidant, the heavy-component formation from the oligomer can be suppressed, so that glycolide can be produced for a long period.

Examples of the phenol-based antioxidant include phenol-based compounds represented by the following formulae (1-1) to (1-5):

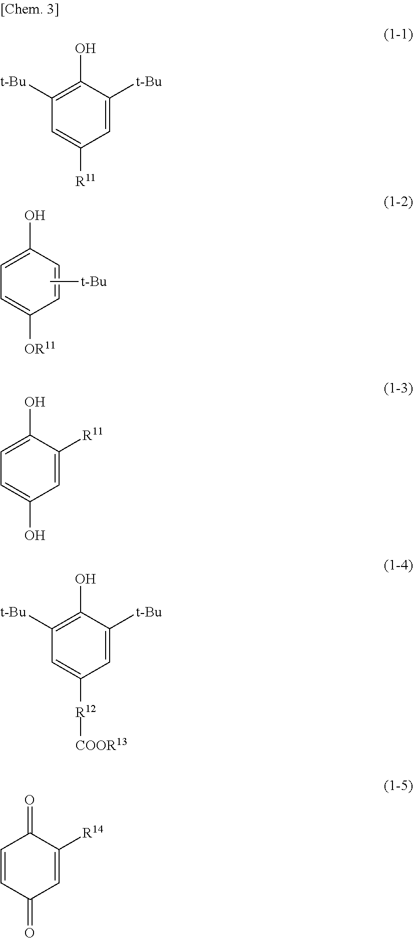

(in the formulae, R¹¹ represents an alkyl group having 1 to 10 (preferably 1 to 5) carbon atoms, R¹² represents an alkylene group having 1 to 5 (preferably 1 to 3) carbon atoms, R¹³ represents an alkyl group having 1 to 30 (preferably 15 to 25) carbon atoms, and R¹⁴ represents hydrogen atom or an alkyl group having 1 to 5 (preferably 1 to 3) carbon atoms);

tocopherol [CAS No: 1406-66-2, molecular weight: 417] represented by the following formula (1-6):

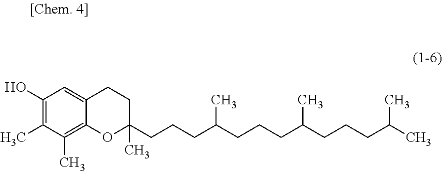 [SMILES: Cc1c(O)cc2c(c1C)OC(C)(CCCC(C)CCCC(C)CCCC(C)C)CC2]

bisphenol-based compounds represented by the following formulae (2-1) and (2-2):

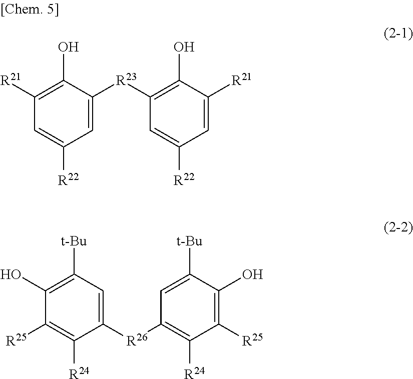

(in the formulae, R²¹ represents t-butyl group or 1-methylcyclohexyl group, R²² represents an alkyl group having 1 to 5 (preferably 1 to 3) carbon atoms, R²³ represents an alkylene group having 1 to 5 (preferably 1 to 3) carbon atoms, R²⁴ and R²⁵ each independently represent hydrogen atom or an alkyl group having 1 to 5 (preferably 1 to 3) carbon atoms, and, preferably, one of R²⁴ and R²⁵ is hydrogen atom, and the other is the alkyl group, and R²⁶ represents sulfur atom, an alkylene group having 1 to 10 (preferably 1 to 5) carbon atoms, or a divalent group having an oxaspiro ring);

triphenol-based compounds represented by the following formula (3):

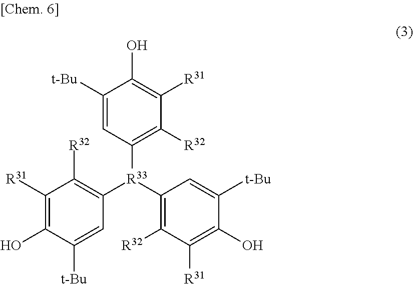

(in the formula, R³¹ represents hydrogen atom or an alkyl group having 1 to 10 (preferably 1 to 5) carbon atoms, R³² represents hydrogen atom or an alkyl group having 1 to 5 (preferably 1 to 3) carbon atoms, provided that one of R³¹ and R³² is hydrogen atom, and the other is the alkyl group, and R³³ represents a trivalent aliphatic hydrocarbon group, a trivalent aromatic group, or a trivalent heterocyclic group);

tetraphenol-based compounds represented by the following formula (4):

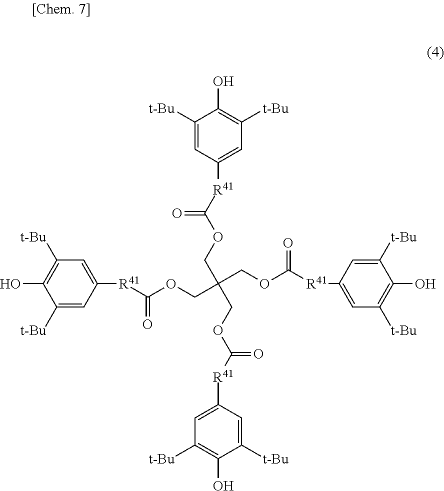

(in the formula, R⁴¹ represents an alkylene group having 1 to 5 (preferably 1 to 3) carbon atoms); and the like.

An example of the divalent group having an oxaspiro ring is the group represented by the following formula (2-2-1):

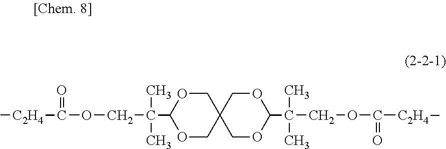

Meanwhile, examples of the trivalent aromatic ring and the trivalent heterocyclic ring include groups represented by the following formulae (3-1) and (3-2):

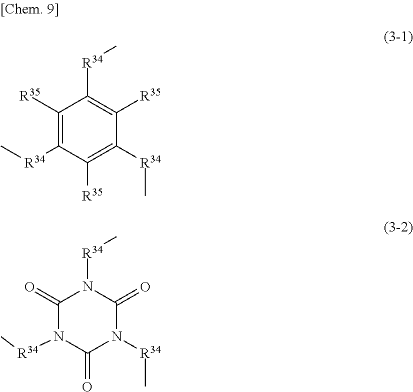

(in the formulae, R³⁴ represents an alkylene group having 1 to 5 (preferably 1 to 3) carbon atoms, and R³⁵ represents an alkyl group having 1 to 5 (preferably 1 to 3) carbon atoms).

In the above-described formulae (1-1) to (1-5), (2-1), (2-2), (3), (3-1), (3-2), and (4), the alkyl group, the alkylene group, the trivalent aliphatic hydrocarbon group may be linear or branched.

Examples of the phenol-based compound represented by the above-described formula (1-1) include 2,6-di-t-butyl-p-cresol [CAS No: 128-37-0, molecular weight: 220] and the like. Examples of the phenol-based compound represented by the above-described formula (1-2) include butylated hydroxyanisole [CAS No: 25013-16-5, molecular weight: 180] and the like. Examples of the phenol-based compound represented by the above-described formula (1-3) include methylhydroquinone [CAS No: 95-71-6, molecular weight: 124] and the like. Examples of the phenol-based compound represented by the above-described formula (1-4) include stearyl-β-(3,5-di-t-butyl-4-hydroxyphenyl)propionate [CAS No: 2082-79-3, molecular weight: 531] and the like. Examples of the phenol-based compound represented by the above-described formula (1-5) include p-benzoquinone [CAS No: 106-51-4, molecular weight: 108], methyl-p-benzoquinone [CAS No: 553-97-9, molecular weight: 122], and the like.

Examples of the bisphenol-based compounds represented by the above-described formula (2-1) include 2,2′-methylenebis(4-methyl-6-t-butylphenol) [CAS No: 119-47-1, molecular weight: 341], 2,2′-methylenebis(4-ethyl-6-t-butylphenol) [CAS No: 88-24-4, molecular weight: 369], 2,2′-dihydroxy-3,3′-di(α-methylcyclohexyl)-5,5′-dimethyldiphenylmethane [CAS No: 77-62-3, molecular weight: 421], and the like. Examples of the bisphenol-based compounds represented by the above-described formula (2-2) include 4,4′-thiobis(3-methyl-6-t-butylphenol) [CAS No: 96-69-5, molecular weight: 359], 4,4′-butylidenebis(3-methyl-6-t-butylphenol) [CAS No: 85-60-9, molecular weight: 383], 3,9-bis[1,1-dimethyl-2-[β-(3-t-butyl-4-hydroxy-5-methylphenyl)propionyloxy]ethyl]2,4,8,10-tetraoxaspiro[5.5]undecane [CAS No: 90498-90-1, molecular weight: 741] and the like.

Examples of the triphenol-based compounds represented by the above-described formula (3) include 1,1,3-tris(2-methyl-4-hydroxy-5-t-butylphenyl)butane [CAS No: 1843-03-4, molecular weight: 545], 1,3,5-trimethyl-2,4,6-tris(3,5-di-t-butyl-4-hydroxybenzyl)benzene [CAS No: 1709-70-2, molecular weight: 775], 1,3,5-tris(3,5-di-t-butyl-4-hydroxybenzyl)-sec-triazine-2,4,6-(1H,3H,5H)trione [CAS No: 27676-62-6, molecular weight: 784], and the like. Examples of the tetraphenol-based compounds represented by the above-described formula (4) include tetrakis[methylene-3-(3,5-di-t-butyl-4-hydroxyphenyl)propionate]methane [CAS No: 6683-19-8, molecular weight: 1178] and the like.

One of these phenol-based antioxidants may be used alone, or two or more thereof may be used in combination. In addition, of these phenol-based antioxidants, phenol-based antioxidants having a molecular weight of 300 or higher are more preferable, phenol-based antioxidants having a molecular weight of 500 or higher are further preferable, and phenol-based antioxidant having a molecular weight of 700 or higher are particularly preferable, from the viewpoint that such phenol-based antioxidants are not easily distilled off during the depolymerization reaction conducted at a high temperature and under a high degree of vacuum.

Examples of phenol-based antioxidants having a molecular weight of 300 to 499 include 2,2′-methylenebis(4-methyl-6-t-butylphenol) [molecular weight: 341], 2,2′-methylenebis(4-ethyl-6-t-butylphenol) [molecular weight: 369], 4,4′-thiobis(3-methyl-6-t-butylphenol) [molecular weight: 359], 4,4′-butylidenebis(3-methyl-6-t-butylphenol) [molecular weight: 383], 2,2′-dihydroxy-3,3′-di(α-methylcyclohexyl)-5,5′-dimethyldiphenylmethane [molecular weight: 421], tocopherol [molecular weight: 417], and the like. Examples of phenol-based antioxidants having a molecular weight of 500 to 699 include stearyl-β-(3,5-di-t-butyl-4-hydroxyphenyl)propionate [molecular weight: 531], 1,1,3-tris(2-methyl-4-hydroxy-5-t-butylphenyl)butane [molecular weight: 545], and the like. Moreover, examples of phenol-based antioxidants having a molecular weight of 700 or higher include 3,9-bis[1,1-dimethyl-2-[β-(3-t-butyl-4-hydroxy-5-methylphenyl)propionyloxy]ethyl]2,4,8,10-tetraoxaspiro[5.5]undecane [molecular weight: 741], 1,3,5-trimethyl-2,4,6-tris(3,5-di-t-butyl-4-hydroxybenzyl)benzene [molecular weight: 775], tetrakis[methylene-3-(3,5-di-t-butyl-4-hydroxyphenyl)propionate]methane [molecular weight: 1178], 1,3,5-tris(3,5-di-t-butyl-4-hydroxybenzyl)-sec-triazine-2,4,6-(1H,3H,5H)trione[molecular weight: 784], and the like.

In the present invention, the amount of the phenol-based antioxidant in the reaction system is preferably 0.5 to 5 parts by mass, and more preferably 1 to 3 parts by mass, relative to 100 parts by mass of the glycolic acid oligomer. If the amount of the phenol-based antioxidant is less than the lower limit, the suppression of the heavy-component formation from the glycolic acid oligomer tends to be insufficient. Meanwhile, an amount of the phenol-based antioxidant exceeding the upper limit tends to be unfavorable in terms of the economy, because of increase in production costs.

Note that, in the depolymerization reaction of the glycolic acid oligomer according to the present invention, it is also possible to use an amine-based antioxidant, a sulfur-based antioxidant, a phosphorus-based antioxidant, or the like, instead of the phenol-based antioxidant. However, the phenol-based antioxidant is used in the present invention from the viewpoint that the phenol-based antioxidant is less likely to have effects such as coloration, denaturation, and degradation on the depolymerization reaction liquid and the glycolide.

Examples of the amine-based antioxidant include piperidine-based compounds represented by the following formula (5):

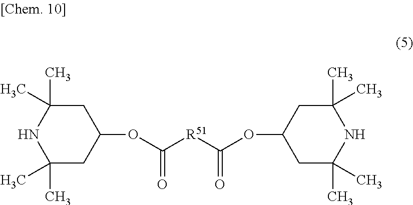

(in the formula, R⁵¹ represents an alkylene group having 1 to 10 carbon atoms). The alkylene group may be linear or branched. Examples of the piperidine-based compounds represented by the above-described formula (5) include bis(2,2,6,6-tetramethyl-4-piperidyl)sebacate [CAS No: 52829-07-9, molecular weight: 481] and the like.

Examples of the sulfur-based antioxidant include sulfide-based compounds represented by the following formula (6):

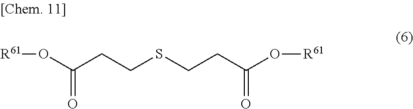

(in the formula, R⁶¹ represents an alkyl group having 1 to 30 (preferably 10 to 20) carbon atoms).
The alkyl group may be linear or branched. Examples of the sulfide-based compounds represented by the above-described formula (6) include dilauryl 3,3′-thiodipropionate [CAS No: 123-28-4, molecular weight: 515], dimyristyl 3,3′-thiodipropionate [CAS No: 16545-54-3, molecular weight: 571], distearyl 3,3′-thiodipropionate [CAS No: 693-36-7, molecular weight: 683], and the like.

Examples of the phosphorus-based antioxidant include phosphite ester compounds represented by the following formulae (7-1) to (7-3):

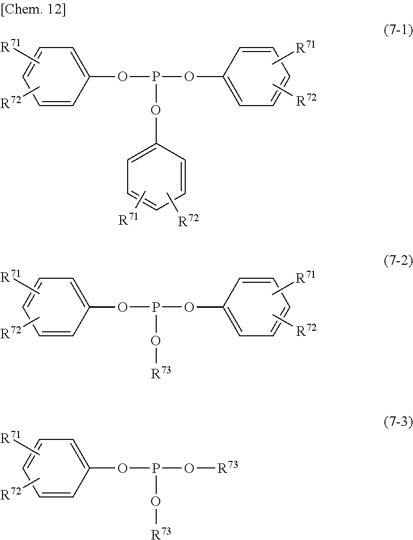

(in the formulae, R⁷¹ and R⁷² each independently represent hydrogen atom or an alkyl group having 1 to 20 (preferably 1 to 10) carbon atoms, and R⁷³ represents an alkyl group having 1 to 20 (preferably 5 to 15) carbon atoms);

phosphite ester compounds represented by the following formula (7-4):

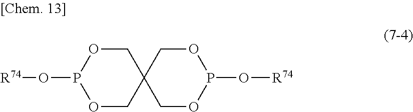

(in the formula, R⁷⁴ represents an alkyl group having 1 to 30 (preferably 15 to 25) carbon atoms or an aryl group having 6 to 30 (preferably 10 to 20) carbon atoms);

phosphite ester compounds represented by the following formula (7-5):

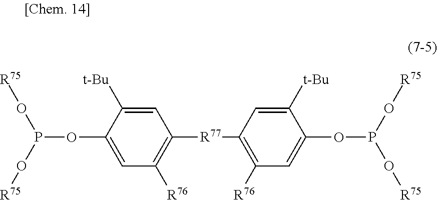

(in the formula, R⁷⁵ represents an alkyl group having 1 to 30 (preferably 10 to 20) carbon atoms, R⁷⁶ represents an alkyl group having 1 to 5 (preferably 1 to 3) carbon atoms, and R⁷⁷ represents an alkylene group having 1 to 10 (preferably 1 to 5) carbon atoms);

phosphite ester compounds represented by the following formula (7-6):

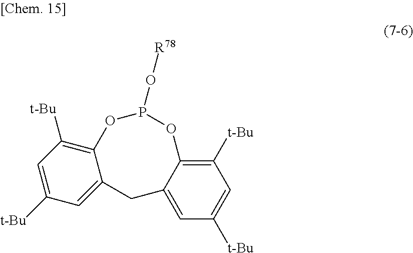

(in the formula, R⁷⁸ represents an alkyl group having 1 to 20 (preferably 5 to 15) carbon atoms); and the like.

The alkyl group and the alkylene group in the above-described formulae (7-1) to (7-6) may be linear or branched.

Examples of the phosphite ester compounds represented by the above-described formula (7-1) include triphenyl phosphite [CAS No: 101-02-0, molecular weight: 310], tris(nonylphenyl)phosphite [CAS No: 26523-78-4, molecular weight: 689], tris(2,4-di-t-butylphenyl)phosphite [CAS No: 31570-04-4, molecular weight: 647], and the like. Examples of the phosphite ester compounds represented by the above-described formula (7-2) include diphenyl isodecyl phosphite [CAS No: 26544-23-0, molecular weight: 374] and the like. Examples of the phosphite ester compounds represented by the above-described formula (7-3) include phenyl diisodecyl phosphite [CAS No: 25550-98-5, molecular weight: 439] and the like.

Examples of the phosphite ester compounds represented by the above-described formula (7-4) include cyclicneopentanetetraylbis(octadecylphosphite) [CAS No: 3806-34-6, molecular weight: 733], cyclicneopentanetetraylbis(2,6-di-t-butyl-4-methylphenyl)phosphite [CAS No: 80693-00-1, molecular weight: 633], and the like. Examples of the phosphite ester compounds represented by the above-described formula (7-5) include 4,4′-butylidene-bis(3-methyl-6-t-butylphenyl ditridecyl)phosphite [CAS No: 13003-12-8, molecular weight: 1240], and the like. Examples of the phosphite ester compounds represented by the above-described formula (7-6) include 2,2-methylenebis(4,6-di-t-butylphenyl)octyl phosphite [CAS No: 126050-54-2, molecular weight: 583] and the like.

(3) Solvent

In the present invention, a solvent is preferably used to improve the depolymerization reactivity of the glycolic acid oligomer. The solvent is preferably a polar organic solvent, and is more preferably a high-boiling point polar organic solvent having a boiling point of 230 to 450° C. The high-boiling point polar organic solvent acts as a solvent in the depolymerization reaction, and also acts as a co-distillation component when the glycolide formed is taken out from the reaction system. Thus, the high-boiling point polar organic solvent makes it possible to prevent the glycolide and the like from adhering to an inner wall of a production line. Accordingly, if the boiling point of the polar organic solvent is lower than the lower limit, the depolymerization reaction temperature cannot be set to a high value, so that the formation rate of glycolide tends to be lowered. Meanwhile, if the boiling point of the polar organic solvent exceeds the upper limit, the polar organic solvent is not easily distilled off during the depolymerization reaction, so that it tends to be difficult to co-distill off the polar organic solvent with the glycolide formed. From such a viewpoint, the boiling point of the high-boiling point polar organic solvent is more preferably 235 to 450° C., further preferably 255 to 430° C., and particularly preferably 280 to 420° C. Here the boiling point of the polar organic solvent in the present invention is a value under normal pressure, and when a boiling point is measured under a reduced pressure, the boiling point is converted to the value under normal pressure.

In addition, the molecular weight of the polar organic solvent is preferably 150 to 450, more preferably 180 to 420, and particularly preferably 200 to 400. If the molecular weight of the high-boiling point polar organic solvent is out of the range, the co-distilling off with glycolide tends to be less likely to occur.

Examples of the high-boiling point polar organic solvent include aromatic dicarboxylic acid diesters, aromatic carboxylic acid esters, aliphatic dicarboxylic acid diesters, polyalkylene glycol diethers, aromatic dicarboxylic acid dialkoxyalkyl esters, aliphatic dicarboxylic acid dialkoxyalkyl esters, polyalkylene glycol diesters, aromatic phosphate esters, and the like. Of these high-boiling point polar organic solvents, aromatic dicarboxylic acid diesters, aromatic carboxylic acid esters, aliphatic dicarboxylic acid diesters, and polyalkylene glycol diethers are preferable, and, from the viewpoint that thermal degradation is less likely to occur, polyalkylene glycol diethers are more preferable. In addition, one of the high-boiling point polar organic solvents may be used alone, or two or more thereof may be used in combination.

Examples of the aromatic dicarboxylic acid diesters include phthalic acid esters such as dibutyl phthalate, dioctyl phthalate, dibenzyl phthalate, and benzyl butyl phthalate. Examples of the aromatic carboxylic acid esters include benzoic acid esters such as benzyl benzoate. Examples of the aliphatic dicarboxylic acid diesters include adipic acid esters such as dioctyl adipate, and sebacic acid esters such as dibutyl sebacate.

Examples of the polyalkylene glycol diethers include compounds represented by the following formula (8):

X¹—O—(R¹—O)ₚ—Y¹ (8)

(in the formula (8), R¹ represents methylene group or a linear or branched alkylene group having 2 to 8 carbon atoms, X¹ represents a hydrocarbon group, Y¹ represents an alkyl group having 2 to 20 carbon atoms or an aryl group having 6 to 20 carbon atoms, p is an integer of 1 or larger, and when p is 2 or larger, a plurality of R¹s may be the same or different from each other).

R¹ in the formula (8) is not particularly limited, as long as R¹ is methylene group or a linear or branched alkylene group having 2 to 8 carbon atoms. R¹ in the formula (8) is preferably an ethylene group, from the viewpoint that the polyalkylene glycol diether represented by the formula (8) is readily available or is easily synthesized.

X¹ in the formula (8) is a hydrocarbon group such as an alkyl group or an aryl group. Of these hydrocarbon groups, X¹ in the formula (8) is preferably a hydrocarbon group having 1 to 20 carbon atoms. If the number of carbon atoms of the hydrocarbon group exceeds the upper limit, the polarity of the polyalkylene glycol diether represented by the formula (8) tends to be low. As a result, the solubility of the glycolic acid oligomer tends to be lowered, and the co-distilling off with the glycolide tends to be difficult. Examples of the alkyl group include methyl group, ethyl group, propyl group, butyl group, pentyl group, hexyl group, heptyl group, octyl group, nonyl group, decyl group, lauryl group, and the like. These alkyl groups may be branched or linear. Examples of the aryl group include phenyl group, naphthyl group, substituted phenyl groups, substituted naphthyl groups, and the like. The substituents of the substituted phenyl groups and the substituted naphthyl groups are each preferably an alkyl group, an alkoxy group, or a halogen atom (Cl, Br, I or the like). The number of the substituents is, for example, 1 to 5, and preferably 1 to 3, when the aryl group is a substituted phenyl group. When multiple substituents are present, the substituents may be the same or different from each other. Note that the substituents play a role of adjusting the boiling point and the polarity of the polyalkylene glycol diether.

Y¹ in the formula (8) is an alkyl group having 2 to 20 carbon atoms or an aryl group having 6 to 20 carbon atoms. If the number of carbon atoms in Y¹ exceeds the upper limit, the polarity of the polyalkylene glycol diether represented by the formula (8) is low. As a result, the solubility of the glycolic acid oligomer is lowered, and the co-distilling off with the glycolide becomes difficult. Meanwhile, if Y¹ is methyl group, the number of carbon atoms in R¹ needs to be large in order that the polyalkylene glycol diether represented by the formula (8) can be a solvent having a high boiling point appropriate for the co-distilling off with the glycolide. However, if such a polyalkylene glycol diether is synthesized, a production process is complicated because the distribution of p is broad, necessitating purification by distillation and the like. Accordingly, polyalkylene glycol diethers whose Y¹ in the formula (8) is methyl group are not preferable, from the viewpoint of carrying out the invention in an industrial scale. Examples of the alkyl group include those described as the examples of the alkyl group which can be X¹, and examples of the aryl group include those described as the examples of the aryl group which can be X¹.

In the formula (8), p is an integer of 1 or larger, and is preferably an integer of 2 or larger. Meanwhile, an upper limit of p is not particularly limited, but is preferably an integer of 8 or smaller, and more preferably an integer of 5 or smaller. If p exceeds the upper limit, the distribution of the degrees of polymerization becomes broad when the polyalkylene glycol diether is synthesized. As a result, it tends to be difficult to isolate a polyalkylene glycol diether having the same p in the formula (8). In addition, when p is 2 or larger, a plurality of R¹s may be the same or different from each other.

Of these polyalkylene glycol diethers, polyalkylene glycol diethers are preferable in which X¹ and Y¹ in the formula (8) are both alkyl groups, and the total number of carbon atoms of X¹ and Y¹ is 3 to 21 (more preferably 6 to 20). In addition, in this case, X¹ and Y¹ may be the same alkyl groups, or different alkyl groups from each other.

Specific examples of the polyalkylene glycol diethers include:

polyethylene glycol dialkyl ethers such as diethylene glycol dibutyl ether, diethylene glycol dihexyl ether, diethylene glycol dioctyl ether, diethylene glycol butyl-2-chlorophenyl ether, triethylene glycol diethyl ether, triethylene glycol dipropyl ether, triethylene glycol dibutyl ether, triethylene glycol dihexyl ether, triethylene glycol dioctyl ether, triethylene glycol butyl octyl ether, triethylene glycol butyl decyl ether, tetraethylene glycol diethyl ether, tetraethylene glycol dipropyl ether, tetraethylene glycol dibutyl ether, tetraethylene glycol dihexyl ether, tetraethylene glycol dioctyl ether, diethylene glycol butyl hexyl ether, diethylene glycol butyl octyl ether, diethylene glycol hexyl octyl ether, triethylene glycol butyl hexyl ether, triethylene glycol hexyl octyl ether, tetraethylene glycol butyl hexyl ether, tetraethylene glycol butyl octyl ether, and tetraethylene glycol hexyl octyl ether;
polyalkylene glycol dialkyl ethers having propyleneoxy groups or butyleneoxy groups with which the ethyleneoxy groups in the above-described polyethylene glycol dialkyl ethers are replaced (for example, polypropylene glycol dialkyl ethers and polybutylene glycol dialkyl ethers); polyethylene glycol alkyl aryl ethers such as diethylene glycol butyl phenyl ether, diethylene glycol hexyl phenyl ether, diethylene glycol octyl phenyl ether, triethylene glycol butyl phenyl ether, triethylene glycol hexyl phenyl ether, triethylene glycol octyl phenyl ether, tetraethylene glycol butyl phenyl ether, tetraethylene glycol hexyl phenyl ether, tetraethylene glycol octyl phenyl ether, and compounds each having an alkyl group, an alkoxy group, a halogen atom, or the like with which a hydrogen atom of the phenyl group in these polyethylene glycol alkyl phenyl ethers is substituted;
polyalkylene glycol alkyl aryl ethers having propyleneoxy groups or butyleneoxy groups with which the ethyleneoxy groups in the above-described polyethylene glycol alkyl aryl ethers are replaced (for example, polypropylene glycol alkyl aryl ethers and polybutylene glycol alkyl aryl ethers);
polyethylene glycol diaryl ethers such as diethylene glycol diphenyl ether, triethylene glycol diphenyl ether, tetraethylene glycol diphenyl ether, and compounds each having an alkyl group, an alkoxy group, a halogen atom, or the like with which a hydrogen atom of a phenyl group in these polyethylene glycol diphenyl ethers is substituted; polyalkylene glycol diaryl ethers having propyleneoxy groups or butyleneoxy groups with which the ethyleneoxy groups in the above-described polyethylene glycol diaryl ethers are replaced (for example, polypropylene glycol diaryl ethers and polybutylene glycol diaryl ethers); and the like. These polyalkylene glycol diethers can be synthesized by the method described in International Publication No. WO02/014303.

Of these polyalkylene glycol diethers, polyalkylene glycol dialkyl ethers are preferable, and diethylene glycol dialkyl ethers, triethylene glycol dialkyl ethers, and tetraethylene glycol dialkyl ethers are more preferable, from the viewpoints that synthesis is easy and thermal degradation is less likely to occur.

In addition, the polyalkylene glycol diether used in the present invention is preferably such that the solubility of glycolide in the polyalkylene glycol diether at 25° C. is 0.1 to 10%. Here the solubility of glycolide is represented by a percentage of the mass (g) of glycolide to the volume (ml) of a polyalkylene glycol diether in a case where glycolide is dissolved in the polyalkylene glycol diether at 25° C. until a saturated state is reached. If the solubility of glycolide is less than the lower limit, the glycolide co-distilled off with the polyalkylene glycol diether tends to deposit at an intermediate portion of a production line, causing blocking of the production line and the like. Meanwhile, if the solubility of glycolide exceeds the upper limit, it may be necessary, in some cases, to isolate the glycolide by, for example, cooling the co-distillate down to 0° C. or below or adding a poor solvent to the co-distillate for the recovery of the co-distilled glycolide.

Examples of the polyalkylene glycol diether in which glycolide has such a predetermined solubility include tetraethylene glycol dibutyl ether (boiling point=340° C., molecular weight=306, solubility of glycolide=4.6%), triethylene glycol butyl octyl ether (boiling point=350° C., molecular weight=350, solubility of glycolide=2.0%), triethylene glycol butyl decyl ether (boiling point=400° C., molecular weight=400, solubility of glycolide=1.3%), diethylene glycol dibutyl ether (boiling point=256° C., molecular weight=218, solubility of glycolide=1.8%), and diethylene glycol butyl 2-chlorophenyl ether (boiling point=345° C., molecular weight=273, solubility of glycolide=1.8%). Of these polyalkylene glycol diethers, tetraethylene glycol dibutyl ether and triethylene glycol butyl octyl ether are more preferable, from the viewpoints of easiness of synthesis, thermal degradation resistance, depolymerization reactivity of the glycolic acid oligomer, recoverability of glycolide, and the like.

In the present invention, the amount of the solvent in the reaction system is preferably 30 to 5000 parts by mass, more preferably 50 to 2000 parts by mass, and particularly preferably 60 to 200 parts by mass, relative to 100 parts by mass of the glycolic acid oligomer. If the amount of the solvent is less than the lower limit, the ratio of a solution phase of the glycolic acid oligomer in the reaction system under the depolymerization temperature condition tends to decrease (the ratio of a melt phase of the glycolic acid oligomer increases), and thereby the depolymerization reactivity of the glycolic acid oligomer tends to be lowered, and the heavy-component formation from the glycolic acid oligomer tends to occur in the melt phase. Meanwhile, if the amount of the solvent exceeds the upper limit, the thermal efficiency during the depolymerization reaction tends to be lowered, and the productivity of the glycolide by the depolymerization reaction tends to be lowered.

(4) Tin Compound

In the present invention, it is preferable to use a tin compound such as tin dichloride, tin tetrachloride, or a tin alkylcarboxylate. The use of such a tin compound suppresses the formation of glycolic acid, a chain dimer, and the like in the depolymerization reaction, making it possible to greatly increase the yield of the glycolide, as compared with a case where no tin compound is used.

One of these tin compounds may be used alone, or two or more thereof may be used in combination. In addition, of these tin compounds, tin dichloride or tin octanoate is preferable, and tin octanoate is more preferable from the viewpoint that the productivity of the glycolide is improved.

In the present invention, the amount of the tin compound in the reaction system is preferably 0.01 to 10 parts by mass, more preferably 0.05 to 2 parts by mass, and particularly preferably 0.1 to 0.5 parts by mass, relative to 100 parts by mass of the glycolic acid oligomer. If the amount of the tin compound is less than the lower limit, the suppression of the formation of glycolic acid, the chain dimer, and the like tends to be insufficient in the depolymerization reaction, and the increase in the yield of glycolide tends to be insufficient. Meanwhile, if the amount of the tin compound exceeds the upper limit, the degradation reaction of the solvent or the solubilizing agent tends to be accelerated, and the degradation products tend to be co-distilled off with glycolide, resulting in a tendency that the purity of glycolide is lowered.

(5) Solubilizing Agent

In the present invention, a solubilizing agent is preferably added in order to improve the dissolution characteristics (solubility and/or dissolution rate) of the glycolic acid oligomer in the solvent (especially, the high-boiling point polar organic solvent). In addition, the addition of the solubilizing agent can also enhance the depolymerization reactivity of the glycolic acid oligomer. The solubilizing agent is preferably a compound satisfying any one or more of the following requirements (1) to (5).

(1) being a non-basic compound. Specifically, basic compounds such as amines, pyridine, and quinoline are not preferable, because these basic compounds may react with the glycolic acid oligomer or the glycolide formed.

(2) being a compound miscible with or soluble in the solvent. The compound may be liquid or solid at ordinary temperature, as long as the compound is miscible with or soluble in the solvent.

(3) being a compound having a boiling point of 180° C. or higher, preferably 200° C. or higher, more preferably 230° C. or higher, and particularly preferably 250° C. or higher. In particular, it is preferable to use, as the solubilizing agent, a compound having a boiling point higher than the boiling point of the solvent used for the depolymerization reaction, because the solubilizing agent is not distilled off at all or the amount of the solubilizing agent distilled off is extremely small during the co-distilling off of glycolide and the solvent. In many cases, favorable results can be obtained by using a compound having a boiling point of 450° C. or higher as the solubilizing agent. Note, however, that alcohols and the like can be suitably used as the solubilizing agent, even when the alcohols and the like are compounds having a boiling point lower than the boiling point of the solvent used for the depolymerization reaction.

(4) being a compound having a functional group, such as OH group, COOH group, or CONH group, for example.

(5) having a higher affinity for the glycolic acid oligomer than the solvent. Here the affinity of the solubilizing agent for the glycolic acid oligomer can be checked as follows. Specifically, a mixture of the glycolic acid oligomer and the solvent is heated at a temperature of 230° C. or higher to form a homogeneous solution phase. Then, the concentration of the glycolic acid oligomer is increased by further adding the glycolic acid oligomer to the solution phase, until the mixture does not form a homogeneous solution phase any more. Then, the solubilizing agent is added thereto, and whether or not a homogeneous solution phase is formed again is visually observed.

In the present invention, a compound satisfying any one or more of these requirements is preferably used as the solubilizing agent. Specifically, it is preferable to use, as the solubilizing agent, at least one non-basic organic compound which is selected from the group consisting of alcohols, phenols, aliphatic carboxylic acids, aliphatic amides, aliphatic imides, polyalkylene glycol diethers having a molecular weight exceeding 450, and sulfonic acids, and which has a boiling point of 180° C. or higher (more preferably 200° C. or higher, further preferably 230° C. or higher, and particularly preferably 250° C. or higher).

Of these solubilizing agents, alcohols are particularly effective. Examples of the alcohols include aliphatic alcohols such as decanol, tridecanol, decanediol, ethylene glycol, propylene glycol, and glycerin; aromatic alcohols such as cresol, chlorophenol, and naphthyl alcohol; polyalkylene glycols; polyalkylene glycol monoethers; and the like. One of these alcohols may be used alone, or two or more thereof may be used in combination.

Moreover, of these alcohols, a polyalkylene glycol monoether represented by the following formula (9) is preferable:

HO—(R²—O)_{q}—X² (9)

(in the formula (9), R² represents methylene group or a linear or branched alkylene group having 2 to 8 carbon atoms, X² represents a hydrocarbon group, q is an integer of 1 or larger, and when q is 2 or larger, a plurality of R²s may be the same or different from each other).
This is because the polyalkylene glycol monoether is hardly distilled off due to the high boiling point. This is also because of the high solubility of the glycolic acid oligomer in the polyalkylene glycol monoether, acceleration of the depolymerization reaction, and a particularly excellent effect of cleaning an inner wall of a reactor.

R² in the formula (9) is not particularly limited, as long as R² is methylene group or a linear or branched alkylene group having 2 to 8 carbon atoms. R² is preferably ethylene group from the viewpoint that the polyalkylene glycol diether represented by the formula (9) is readily available or is easily synthesized. In addition, X² in the formula (9) is a hydrocarbon group such as an alkyl group or an aryl group. Of these, X² is preferably a hydrocarbon group having 1 to 18 carbon atoms, and more preferably a hydrocarbon group having 6 to 18 carbon atoms.

Of these polyalkylene glycol monoethers, preferred are polyethylene glycol monoalkyl ethers such as polyethylene glycol monomethyl ether, polyethylene glycol monoethyl ether, polyethylene glycol monopropyl ether, polyethylene glycol monobutyl ether, polyethylene glycol monohexyl ether, polyethylene glycol monooctyl ether, polyethylene glycol monodecyl ether, and polyethylene glycol monolauryl ether; and polyalkylene glycol monoalkyl ethers having propyleneoxy groups or butyleneoxy groups with which the ethyleneoxy groups in the above-described polyethylene glycol monoalkyl ethers are replaced (for example, polypropylene glycol monoalkyl ethers and polybutylene glycol monoalkyl ethers), and more preferred are polyethylene glycol monohexyl ether, polyethylene glycol monooctyl ether, polyethylene glycol monodecyl ether, and polyethylene glycol monolauryl ether; and polyalkylene glycol monoethers having propyleneoxy groups or butyleneoxy groups with which the ethyleneoxy groups in the above-described polyethylene glycol monoalkyl ethers are replaced. One of these polyalkylene glycol monoethers may be used alone, or two or more thereof may be used in combination.

Moreover, examples of other preferred alcohols include polyalkylene glycols represented by the following formula (10):

HO—(R³—O)ᵣ—H (10)

(in the formula (10), R³ represents methylene group or a linear or branched alkylene group having 2 to 8 carbon atoms, r is an integer of 1 or larger, and when r is 2 or larger, a plurality of R³s may be the same or different from each other).

R³ in the formula (10) is not particularly limited, as long as R³ is methylene group or a linear or branched alkylene group having 2 to 8 carbon atoms. R³ is preferably ethylene group from the viewpoint that the polyalkylene glycol represented by the formula (10) is readily available or is easily synthesized.

Examples of the polyalkylene glycols include polyethylene glycol, polypropylene glycol, polybutylene glycol, and the like. One of these polyalkylene glycols may be used alone, or two or more thereof may be used in combination.

Examples of the polyalkylene glycol diethers having a molecular weight exceeding 450 each used as the solubilizing agent include polyethylene glycol dimethyl ether #500 (average molecular weight: 500), polyethylene glycol dimethyl ether #2000 (average molecular weight: 2000), and the like. If the molecular weight is the lower limit or smaller, the solubilizing agent is also distilled off together with the distilling-off of glycolide during the depolymerization reaction, resulting in a tendency that the solubility of the glycolic acid oligomer in the mixture according to the present invention is lowered.

Note that the action of the solubilizing agent in the depolymerization reaction of the glycolic acid oligomer is not sufficiently clarified as of now; however, the present inventors presume as follows. Specifically, the solubilizing agent presumably exerts 1) an action of converting the glycolic acid oligomer to a more soluble material (state) by reacting with terminals of the glycolic acid oligomer, 2) an action of converting the glycolic acid oligomer to a more soluble material by acting on internal portions of molecular chains of the glycolic acid oligomer and cleaving the molecular chains to thereby adjust the molecular weight of the glycolic acid oligomer, 3) an action of increasing the solubility of the glycolic acid oligomer by changing the polarity of the solvent system to thereby increase the hydrophilicity of the entire solvent system, 4) an action of emulsifying and dispersing the glycolic acid oligomer, 5) an action of increasing the number of depolymerization reaction points by binding to one of the terminals of the glycolic acid oligomer, 6) an action of increasing the number of depolymerization reaction points by acting on internal portions of the glycolic acid oligomer to thereby cleave the glycolic acid oligomer and to bind to terminals of the cleaved molecular chains, and 7) a combined action of these actions.

In the present invention, the amount of the solubilizing agent in the reaction system is preferably 0.1 to 500 parts by mass and more preferably 1 to 300 parts by mass, relative to 100 parts by mass of the glycolic acid oligomer. If the amount of the solubilizing agent is less than the lower limit, the dissolution characteristics of the glycolic acid oligomer in the solvent (especially, the high-boiling point polar organic solvent) may be lowered. Meanwhile, an amount of the solubilizing agent exceeding the upper limit tends to be not preferable in terms of economy, because the recovery of the solubilizing agent requires a lot of costs.

<Method for Producing Glycolide>

In a method for producing glycolide of the present invention, the glycolic acid oligomer is depolymerized in the presence of the phenol-based antioxidant. The depolymerization is preferably conducted in the solvent. This makes it possible to improve the formation rate and the volatilization rate of glycolide. In addition, it is also preferable to conduct the depolymerization in the presence of a tin compound or in the presence of a solubilizing agent, or under a condition where these are combined. A method for depolymerizing a glycolic acid oligomer in a solvent in the presence of a phenol-based antioxidant will be described in detail below.

(Dissolving Step)

First, the glycolic acid oligomer, the phenol-based antioxidant, and the solvent are mixed with each other. The obtained mixture is heated to dissolve the glycolic acid oligomer and the phenol-based antioxidant in the solvent. Here, it is preferable to mix a solubilizing agent with the mixture. This improves the solubility of the glycolic acid oligomer in the solvent, making it possible to greatly improve the formation rate and the volatilization rate of glycolide. In addition, if necessary, a tin compound may be mixed. This can increase the yield of glycolide.

A temperature to which the mixture is heated is preferably 200 to 350° C., more preferably 210 to 310° C., particularly preferably 220 to 300° C., and most preferably 230 to 290° C. If the heating temperature is lower than the lower limit, the glycolic acid oligomer is not easily dissolved in the solvent, and hence a homogeneous solution is difficult to obtain, so that the depolymerization reactivity of the glycolic acid oligomer tends to decrease. Meanwhile, if the heating temperature exceeds the upper limit, the heavy-component formation from the glycolic acid oligomer tends to occur.

In addition, the heating of the mixture may be conducted under normal pressure or reduced pressure, and is preferably conducted under a reduced pressure of 0.1 to 90 kPa (more preferably 1 to 30 kPa, particularly preferably 1.5 to 20 kPa, and most preferably 2 to 10 kPa). Moreover, it is also preferable to conduct the heating under an inert gas atmosphere.

When the glycolic acid oligomer is dissolved in the solvent, it is preferable to form a homogeneous solution phase. However, the melt phase of the glycolic acid oligomer may be remained, as long as the ratio of the remaining melt phase of the glycolic acid oligomer is 0.5 or less. The term “the ratio of the remaining melt phase” means a ratio represented by b/a, where a (ml) is the volume of the melt phase of a glycolic acid oligomer formed when a predetermined amount of the glycolic acid oligomer is added to a solvent, such as liquid paraffin, which is substantially incapable of dissolving the glycolic acid oligomer, and then the resultant is heated until a temperature at which the glycolic acid oligomer is depolymerized; and b (ml) is the volume of the melt phase of the glycolic acid oligomer formed when the same amount of the glycolic acid oligomer is added to a solvent to be actually used, and the resultant is heated until the temperature at which the glycolic acid oligomer is depolymerized. The ratio of the remaining melt phase is more preferably 0.3 or less, particularly preferably 0.1 or less, and most preferably substantially zero. If the ratio of the remaining melt phase exceeds the upper limit, the distilling-off of the glycolide formed becomes less likely to occur, and the heavy-component formation from the glycolic acid oligomer tends to occur in the melt phase.

(Depolymerization Step)

Next, the heating of the thus prepared solution phase (the resultant obtained by substantially homogeneously dissolving the glycolic acid oligomer, the phenol-based antioxidant, and if necessary, the solubilizing agent and the tin compound in the solvent) is further continued. Thus, the glycolic acid oligomer is depolymerized in the solution phase, and glycolide is formed. In the present invention, since the glycolic acid oligomer is depolymerized in the presence of a phenol-based antioxidant, the heavy-component formation from the oligomer is suppressed, so that the glycolide can be produced for a long period.

Preferred conditions of the temperature, the pressure, and the like for the depolymerization reaction are the same as the preferred conditions for the dissolving step. In addition, the heating conditions in the depolymerization step may be the same as or different from the heating conditions in the dissolving step. In particular, the pressure is preferably set as low as possible, from the viewpoint that the depolymerization reaction temperature is lowered, and the recovery ratio of the solvent is improved. In general, the heating is conducted at a pressure lower than the pressure in the dissolving step.

(Distilling-Off Step)

Next, the thus formed glycolide is distilled off together with the solvent. This makes it possible to suppress the adherence of glycolide to an inner wall of a production line, and to prevent the blocking of the line. In addition, the depolymerization reaction is a reversible reaction. Hence, the depolymerization reaction of the glycolic acid oligomer proceeds efficiently by the distilling-off of the glycolide from the reaction system. Particularly when the depolymerization reaction is conducted under a reduced pressure, the glycolide is easily distilled off, and the depolymerization reaction proceeds more efficiently.

When glycolide is continuously produced by the production method of the present invention, the glycolic acid oligomer in an amount corresponding to the amount of the glycolide distilled off is preferably supplied continuously or intermittently to the depolymerization reaction system. Here, it is necessary to supply the glycolic acid oligomer such that the state where the glycolic acid oligomer is homogeneously dissolved in the solvent can be kept. In addition, when any of the phenol-based antioxidant, the solvent, the solubilizing agent, and the tin compound is distilled off, the corresponding one of the phenol-based antioxidant, the solvent, the solubilizing agent, and the tin compound in an amount corresponding to the amount distilled off is preferably added continuously or intermittently to the depolymerization reaction system. Note that the phenol-based antioxidant, the solvent, the solubilizing agent, and the tin compound to be supplied may be fresh ones, or those recovered in the following recovery step may be reused.

(Recovery Step)

The glycolide distilled off together with the solvent as described above can be recovered by a method described in Japanese Unexamined Patent Application Publication No. 2004-523596 or International Publication No. WO02/014303. For example, the glycolide can be recovered by cooling a co-distillate of the glycolide and the solvent, and if necessary, adding a poor solvent to solidify and precipitate the same. Alternatively, as described in International Publication No. WO02/014303, the glycolide can be recovered also by phase separation, when a solvent having an excellent heat stability is used.

### EXAMPLES

Hereinafter, the present invention will be described more specifically on the basis of Examples and Comparative Examples. However, the present invention is not limited to Examples below. Note that the melting point of a glycolic acid oligomer was measured by the following method.

<Melting Point of Glycolic Acid Oligomer>

The melting point was measured by using a differential scanning calorimeter (DSC) under an inert gas atmosphere and under a condition of a rate of temperature rise of 10° C./minute.

### Preparation Example 1

Into a 1-liter separable flask, 1 kg of a 70% glycolic acid aqueous solution (manufactured by DuPont, industrial grade) was put, and heated at normal pressure with stirring to raise the temperature from room temperature to 220° C. over 4 hours. During this period, a condensation reaction was conducted, while water produced was removed by distillation. Next, the pressure inside the flask was gradually reduced from normal pressure to 2 kPa over 1 hour, and then the condensation reaction was continued by heating at 220° C. for 3 hours. After that, low boiling components such as the unreacted raw material were removed by distillation. Thus, 480 g of glycolic acid oligomer (GAO) was obtained. The glycolic acid oligomer had a melting point of 211° C.

### Example 1

Into a 100-ml pressure vessel, 4.57 g of the glycolic acid oligomer (GAO) obtained in the Preparation Example 1, 2.86 g of tetraethylene glycol dibutyl ether (TEG-DB, boiling point: 340° C., molecular weight: 306, solubility of glycolide: 4.6%) as a solvent, 2.54 g of octyl triethylene glycol (OTEG) as a solubilizing agent, 0.071 g of tin dichloride (SnCl₂) dihydrate as a catalyst, and 100 mg of 1,3,5-trimethyl-2,4,6-tris(3,5-di-t-butyl-4-hydroxybenzyl)benzene (“Adeka Stab AO-330” manufactured by ADEKA, molecular weight: 775) as a phenol-based antioxidant were supplied, and heated up to 260° C. Thus, a homogeneous solution was prepared.

A depolymerization reaction was conducted by allowing the solution to stand for one day with heating at 260° C. Thus, glycolide was synthesized. An alkaline degradation treatment was conducted by adding 5 ml of 0.1 g/ml sodium hydroxide aqueous solution to 1 g of the obtained solution, and heating at 95° C. for 5 hours. The solution was filtered, and the residue (alkaline degradation insoluble matters) was vacuum dried at 60° C. for 2 days. Then, the mass of the alkaline degradation insoluble matters was measured to determine the concentration of the alkaline degradation insoluble matters in the solution after completion of the reaction. As a result, the concentration was 3.2% by mass.

### Comparative Example 1

Glycolide was synthesized, and the concentration of alkaline degradation insoluble matters in the solution after completion of the reaction was determined in the same manner as in Example 1, except that no phenol-based antioxidant was used. As a result, the concentration was 11.6% by mass.

As is apparent from the results described above, it was found that the heavy-component formation from the glycolic acid oligomer was successfully suppressed by adding the phenol-based antioxidant in the case where glycolide was produced by depolymerization of the glycolic acid oligomer.

### INDUSTRIAL APPLICABILITY

As described above, according to the present invention, the heavy-component formation from the glycolic acid oligomer can be suppressed in a depolymerization reaction of the oligomer.

Hence, the method for producing glycolide of the present invention is useful as an industrially advantageous method for producing glycolide, because problems such as blocking of the production line are less likely to occur, so that glycolide can be produced stably for a long period (for example, 10 days or more, preferably 20 days or more, and more preferably 50 days or more).

1. A method for producing glycolide, comprising depolymerizing a glycolic acid oligomer in the presence of a phenol-based antioxidant.

2. The method for producing glycolide according to claim 1, wherein the phenol-based antioxidant is a phenol-based antioxidant having a molecular weight of 300 or higher.

3. The method for producing glycolide according to claim 1, wherein the glycolic acid oligomer is depolymerized in a solvent.

4. The method for producing glycolide according to claim 3, wherein the solvent is a high-boiling point polar organic solvent having a boiling point of 230 to 450° C.

5. The method for producing glycolide according to claim 3, wherein the glycolide obtained by the depolymerization and the solvent are co-distilled off.

6. The method for producing glycolide according to claim 1, wherein the glycolic acid oligomer is depolymerized in the presence of a tin compound.

7. The method for producing glycolide according to claim 2, wherein the glycolic acid oligomer is depolymerized in a solvent.

8. The method for producing glycolide according to claim 7, wherein the solvent is a high-boiling point polar organic solvent having a boiling point of 230 to 450° C.

9. The method for producing glycolide according to claim 4, wherein the glycolide obtained by the depolymerization and the solvent are co-distilled off.

10. The method for producing glycolide according to claim 7, wherein the glycolide obtained by the depolymerization and the solvent are co-distilled off.

11. The method for producing glycolide according to claim 8, wherein the glycolide obtained by the depolymerization and the solvent are co-distilled off.

12. The method for producing glycolide according to claim 2, wherein the glycolic acid oligomer is depolymerized in the presence of a tin compound.

13. The method for producing glycolide according to claim 3, wherein the glycolic acid oligomer is depolymerized in the presence of a tin compound.

14. The method for producing glycolide according to claim 4, wherein the glycolic acid oligomer is depolymerized in the presence of a tin compound.

15. The method for producing glycolide according to claim 5, wherein the glycolic acid oligomer is depolymerized in the presence of a tin compound.

16. The method for producing glycolide according to claim 7, wherein the glycolic acid oligomer is depolymerized in the presence of a tin compound.

17. The method for producing glycolide according to claim 8, wherein the glycolic acid oligomer is depolymerized in the presence of a tin compound.

18. The method for producing glycolide according to claim 9, wherein the glycolic acid oligomer is depolymerized in the presence of a tin compound.

19. The method for producing glycolide according to claim 10, wherein the glycolic acid oligomer is depolymerized in the presence of a tin compound.

20. The method for producing glycolide according to claim 11, wherein the glycolic acid oligomer is depolymerized in the presence of a tin compound.
